(19) United States
(12) Patent Application Publication
(10) Pub. No.: US 2026/0013880 A1
(43) Pub. Date: Jan. 15, 2026

(54) SYSTEMS, DEVICES, AND METHODS FOR PASSING IMPLANTS, SUTURES, OR TISSUES THROUGH BONE TUNNELS

(71) Applicant: AESCLEPIUS CORPORATION, La Jolla, CA (US)

(72) Inventors: Adam H. Hsieh, La Jolla, CA (US); Joe Ty Lin, San Diego, CA (US); Jacob Lev Rudelson, San Diego, CA (US)

(21) Appl. No.: 19/084,456

(22) Filed: Mar. 19, 2025

Related U.S. Application Data
(63) Continuation of application No. PCT/US23/33277, filed on Sep. 20, 2023.
(60) Provisional application No. 63/408,703, filed on Sep. 21, 2022.
(60) Provisional application No. 63/417,019, filed on Oct. 18, 2022.
(60) Provisional application No. 63/432,127, filed on Dec. 13, 2022.
(60) Provisional application No. 63/539,470, filed on Sep. 20, 2023.

(51) Int. Cl. A61B 17/17; A61B 17/00; A61B 17/16; A61B 17/56

(52) U.S. Cl. CPC A61B 17/1732; A61B 17/1615; A61B 17/1775; A61B 2017/00407; A61B 2017/00862; A61B 2017/00867; A61B 2017/564

(57) ABSTRACT

Systems, methods, and devices for passing implants, sutures, or tissue through bone tunnels are provided. For example, disclosed herein are embodiments of a device for capturing and retrieving an implant, a suture, or tissue, wherein the device comprises a housing, a guide shaft, and a filament configured to slide freely within the guide shaft, and wherein the filament forms a bight configured to enlarge or shrink by advancing or retracting the filament.

### CROSS-REFERENCE TO RELATED APPLICATIONS

This application is a continuation of International Patent Application No. PCT/US2023/033277, filed Sep. 20, 2023, which claims priority to and the benefit of U.S. Provisional Patent Application No. 63/539,470 filed Sep. 20, 2023, U.S. Provisional Patent Application No. 63/432,127 filed Dec. 13, 2022, U.S. Provisional Patent Application No. 63/417,019 filed Oct. 18, 2022, and U.S. Provisional Patent Application No. 63/408,703 filed Sep. 21, 2022, all of which are incorporated by reference herein in their entireties for all purposes.

### FIELD

The subject matter described herein relates generally to systems, devices, and methods for passing implants, sutures, or tissue through bone tunnels. In particular, described herein are embodiments of systems configured to follow straight or curvilinear tunnels in bone, as well as methods and devices relating thereto.

### BACKGROUND

Joint arthropathies (diseases that compromise joint function) are part of a steadily growing worldwide trend in chronic musculoskeletal disorders. In 2012, the Bone and Joint Initiative published findings that one out of every two Americans were diagnosed with musculoskeletal conditions, accounting for hundreds of billions of dollars in costs, which continue to grow annually. In 2018, the World Health Organization (WHO) identified the second largest contributor to global disability as musculoskeletal conditions. The increasing number of afflicted people and a continued rise in treatment costs point to a critical need for new technologies that provide more effective solutions to manage musculoskeletal ailments.

Joint arthropathies caused by soft tissue damage (e.g., tendon, ligament, and/or fibrocartilage tears) make up the majority of cases within the broader category of musculoskeletal conditions. Shoulder pain stands among the most common musculoskeletal complaints worldwide, with rotator cuff tears being the leading cause of shoulder disability. Other types of ligament, tendon, and fibrocartilage injuries, such as labral tears, meniscus root tears, Achilles tendon avulsions, anterior cruciate ligament (ACL) ruptures, and lateral ankle ligament tears, among others, are somewhat less prevalent, but no less debilitating. Most of these injuries, whether due to tear size or lack of responsiveness to conservative treatment (e.g., physical therapy), require primary surgical repair. In 2014, the United States Agency for Healthcare Research and Quality (AHRQ) reported over 1.8 million invasive, therapeutic surgeries involving “muscle, tendon, soft tissue operating room procedures” and “incision or fusion of joint, or destruction of joint lesion” in the United States, which equates to 8.3% of the roughly 21.7 million total ambulatory and inpatient surgical procedures.

The goal of such repairs is to re-establish the position and direction of force transmission in these tissues in order to restore stability and motion to their respective joints. For soft tissue injuries, this can be achieved by re-attaching the torn areas of soft tissue (e.g., tendon, ligament, and/or fibrocartilage)—which naturally pull away from their anatomic insertion site upon injury—using a fixation method to create a stable connection and close contact between tissue and bone so that the interface can heal over time.

In some soft tissue surgical repair techniques, a bone tunnel is required either for the insertion of an implant, suture, or tissue. For instance, ACL reconstructions often employ the use of straight bone tunnels for both femoral and tibial fixation of graft tissue using interference screws and/or bone plugs. As another example, rotator cuff repairs may utilize a transosseous approach involving the creation of curvilinear or piece-wise linear bone tunnels through which sutures are passed to pull the tom tendon back to the bone.

Thus, needs exist for systems, devices, and methods to pass or retrieve an implant, suture, or tissue through straight or curvilinear bone tunnels, where said systems, devices, and methods can effectively navigate through said tunnels, particularly those having small diameters, and that also have the means to secure a target object.

### SUMMARY

Provided herein are example embodiments of systems, devices and methods for retrieving an implant, suture, or tissue through straight or curvilinear bone tunnels.

According to some embodiments, a device for capturing and retrieving an implant, a suture, or tissue is provided, wherein the device comprises a housing rigidly attached to a guide shaft, a filament configured to slide freely within the guide shaft, and wherein a plurality of ends of the filament passes through the guide shaft to form a bight, wherein the bight comprises an opening configured to be enlarged or shrunken by respectively advancing and/or retracting the filament, and wherein the bight comprises a curved configuration.

According to another embodiment, an apparatus for clamping a bone material is provided, wherein the apparatus comprises a plurality of finger rings, each of which is coupled with a corresponding shank of a plurality of shanks, wherein the plurality of shanks is configured to transmit a manual force received from the plurality of finger rings; a hinge coupled with the plurality of shanks, wherein the hinge comprises a fulcrum configured to create a scissoring action by the plurality of shanks; a plurality of jaw elements disposed at a plurality of distal portions of the plurality of shanks, wherein the plurality of jaw elements is configured to transmit the manual force from the plurality of shanks to the bone material; a plurality of serrated curved arms coupled with a plurality of distal portions of the plurality of jaw elements, the plurality of serrated curved arms configured to cradle and secure the bone material; and a drill guide configured to control a position and a trajectory of a tunneling instrument for boring one or more tunnels through the bone material.

Many of the embodiments provided herein provide improved methods and techniques for passing implants, sutures, or tissue through bone tunnels. Other improvements and advantages are provided as well. The various configurations of these devices are described in detail by way of the embodiments which are only examples.

Other systems, devices, methods, features and advantages of the subject matter described herein will be or will become apparent to one with skill in the art upon examination of the following figures and detailed description. Where a method is described and claimed herein, analyte monitoring systems comprising means for performing each of the steps of the method are also expressly disclosed and provided. Moreover, computer programs, computer program products and computer readable media for implementing the steps of the method are also disclosed and provided. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are perspective views of an example embodiment of a device, in extended and retracted configurations, respectively, used to retrieve an implant, suture, or tissue, in part or in its entirety.

FIGS. 2A-G are progressive diagrammatic views of an example embodiment of a device, in various stages of operation, used to retrieve an implant, suture, or tissue through a curved bone tunnel.

FIGS. 3A-H are progressive diagrammatic views of an example embodiment of a device, in various stages of operation, used to retrieve an implant, suture, or tissue through a straight bone tunnel.

FIGS. 4A-J are progressive diagrammatic views of an example method for using a clamping device with drill guides to create straight bone tunnels, and then using a device to pass an implant, suture, or tissue through the bone tunnels.

FIGS. 5A-B are perspective views of another example embodiment of a device, in extended and retracted configurations, respectively, used to retrieve an implant, suture, or tissue, in part or in its entirety. FIGS. 5C-5D are side, top, and front views, respectively, of example embodiments of a device used to retrieve an implant, suture, or tissue, in part or in its entirety.

FIGS. 6A-C are diagrams of an embodiment of a device, in various perspective and orthogonal views of closed and open configurations, used to clamp bone in a stable and secure manner.

FIGS. 7A-J are progressive diagrammatic views of another example method for using a clamping device with drill guides to create straight bone tunnels, and then using a device to pass an implant, suture, or tissue through the bone tunnels.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure include systems, devices, and methods for retrieving an implant, suture, or tissue, and any part thereof, through straight or curvilinear tunnels within bone, cartilage, fibrocartilage, tendon, or ligament. In certain embodiments, an implant, suture, or tissue retrieval device (hereafter referred to as a “retrieval device”) includes a retractable tube that can be made of a superelastic material. One example of a superelastic material is nickel-titanium alloy, also known as nitinol According to the embodiments, the superelastic tube can be retracted into a larger caliber curved or straight guide shaft. After the guide shaft is positioned, the retractable superelastic tube can be extended from the guide shaft, can be rotated to adjust trajectory, and will assume its shape set geometric configuration. Passing through the retractable superelastic tube is a segment of filament whose two ends are either within the retractable superelastic tube, or extend through and back out of the retractable superelastic tube, thereby creating a bight at the tip of the extending end of the retractable superelastic tube. The bight can be lengthened or shortened, respectively, by applying a pushing or pulling force to one or both ends of the filament. This pushing and pulling of the filament loosens and tightens the bight around an implant, suture, or tissue Retraction of the superelastic tube into the guide shaft, along with any subsequent repositioning of the guide shaft, allows the captured implant, suture, or tissue to be pulled through the straight or curvilinear tunnel within bone, cartilage, fibrocartilage, tendon, or ligament.

For each and every embodiment of a method disclosed herein, systems and devices capable of performing each of those embodiments are covered within the scope of the present disclosure. For example, embodiments of retrieval devices are disclosed, and these devices can each have one or more internal propulsion mechanisms.

### Example Embodiments of Retrieval Devices and Methods Relating Thereto

Example embodiments of retrieval devices for capturing and retrieving an implant, suture, or tissue, and methods relating thereto, will now be described.

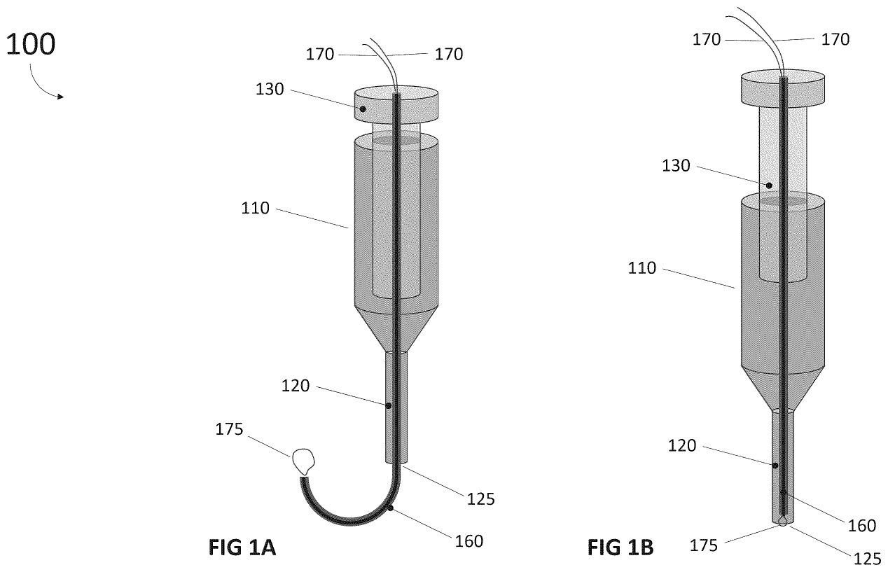

FIGS. 1A and 1B depict perspective views of an embodiment of retrieval device 100 in extended and retracted configurations, respectively. According to one aspect of many embodiments, retrieval device 100 can comprise a housing 110, a guide shaft 120, and a plunger 130. Housing 110 is rigidly attached to guide shaft 120, and plunger 130 is configured to slide and rotate axially within housing 110. A tube 160, possessing a curved geometry on one end, is rigidly attached to plunger 130 and is configured to slide through a channel within guide shaft 120 when a force is applied to plunger 130. In some embodiments, tube 160 can comprise a superelastic material, one of numerous examples of which include nickel-titanium alloy, also known as nitinol. Within tube 160 is a filament 170 whose two ends extend through and back out of tube 160 and plunger 130, thereby creating an bight 175 at the tip of the extending end of tube 160. The axial sliding action of plunger 130 within housing 110 causes tube 160 to retract into and extend out from guide shaft 120 via shaft exit point 125. Pushing and pulling forces applied to one or both ends of filament 170 causes it to slide within plunger 130 and tube 160, thereby enlarging or shrinking the size of bight 175.

It should be noted that although guide shaft 120 is depicted as being straight, those of skill in the art will appreciate that the guide shaft can be curved, can be of any material composition, metallic or non-metallic, natural or synthetic, including a superelastic material. Additionally, tube 160 is depicted to possess a single curve at one end, but those of skill in the art will recognize that tube 160 can possess multiple curves of different curvatures and in varying planes or can be straight with no curves. Filament 170 can be of any material metallic or non-metallic, natural or synthetic, including a superelastic material. Those of skill in the art will appreciate that the sliding of plunger 130 within housing 110, and the sliding of filament 170 within tube 160 can be achieved either by a manual or automated mechanism, internal or external to the device.

One of many methods for using retrieval device 100, whether in arthroscopic or in open surgery, comprises a sequence of general steps, as follows. In the initial configuration of device 100, filament 170 can be pulled back through tube 160 and plunger 130, thereby minimizing the size of bight 175 at the tip of tube 160. Plunger 130 can be completely pulled back, such that tube 160 is fully retracted inside guide shaft 120. Device 100 can then be positioned to place shaft exit point 125 at the desired location in the desired orientation. Once device 100 is satisfactorily positioned, plunger 130 can be pushed toward guide shaft 120, which will extend tube 160 through shaft exit point 125. Plunger 130 can also be rotated to adjust the trajectory by which tube 160 extends into or through a bone tunnel, a soft tissue, or a joint space. Extension and rotation actions of tube 160 can be terminated when bight 175 at the tip of tube 160 reaches the desired location. A pushing force can then be applied to the two ends of filament 170, causing the enlargement of bight 175 and enabling the insertion of an implant, suture, or tissue into the space defined by bight 175. A pulling force is then applied to the two ends of filament 170, resulting in the tightening of bight 175 around the implant, suture, or tissue. Once captured, the implant, suture, or tissue can be passed through the bone tunnel, soft tissue, or joint space in question by applying a pulling force to plunger 130. This will cause the captured implant, suture, or tissue to be pulled by tube 160 toward shaft exit point 125, as tube 160 retracts into guide shaft 120. Device 100 can then be moved to position the captured implant, suture, or tissue to a different location. A pushing force applied to filament 170 can be used to release the captured implant, suture, or tissue.

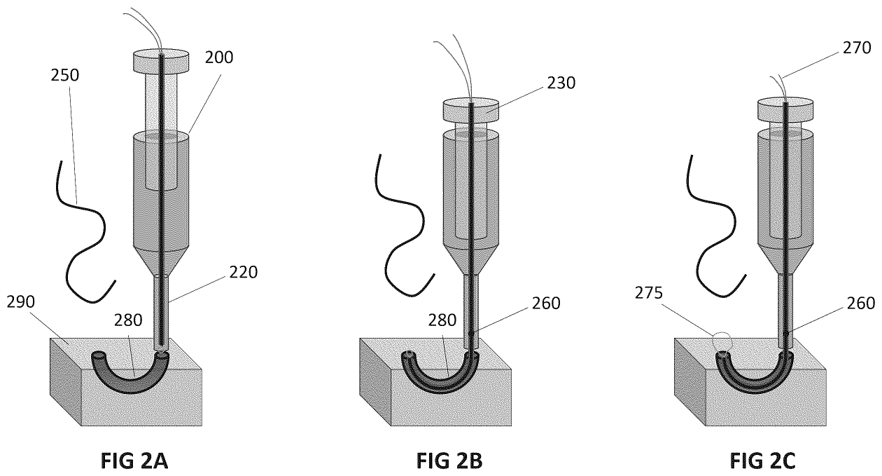

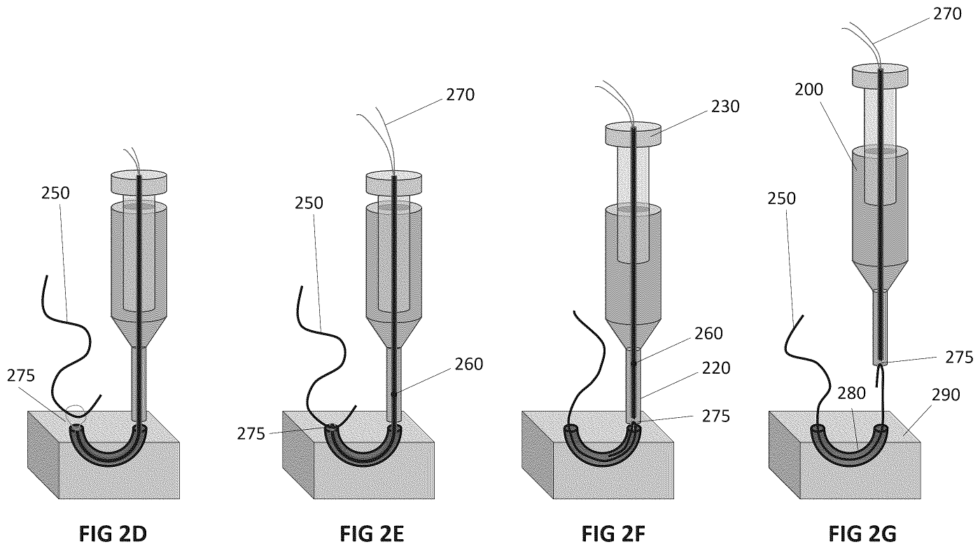

FIGS. 2A-G depict the use of retrieval device 200 in an example application. Here, the retrieval device 200 is shown to retrieve one end of an implant, suture, or tissue 250 through an existing curved tunnel 280 within bone 290. In FIG. 2A, the retrieval device 200, in fully retracted configuration, is positioned with the guide shaft 220 near one end of curved bone tunnel 280. Implant, suture, or tissue 250 remains outside of the bone 290. In FIG. 2B, downward force can be applied to plunger 230, whether by a manual or automated mechanism internal or external to the device, in order to cause tube 260, which can be comprised of superelastic material, to extend into the curved bone tunnel 280. In FIG. 2C, the two ends of filament 270 are pushed downward through tube 260, whether by a manual or automated mechanism internal or external to the device, in order to cause bight 275 to enlarge. In FIG. 2D, the implant, suture, or tissue 250 is manipulated by the user to cause one part of the implant, suture, or tissue to become pass through the opening formed by bight 275. In FIG. 2E, the two ends of filament 270 are pulled upward through tube 260, whether by a manual or automated mechanism internal or external to the device, in order to cause bight 275 to tighten around part of the implant, suture, or tissue 250 that was placed through the bight opening. In FIG. 2F, upward force is applied to plunger 230, whether by a manual or automated mechanism internal or external to the device, causing plunger 230 to slide upward and retracting tube 260 back into guide shaft 220, while bight 275 remains tightened around implant, suture, or tissue 250. In FIG. 2G, the retrieval device 200 is moved away from bone 290, while bight 275 remains tightened around implant, suture, or tissue 250, in order to pull one end of implant, suture, or tissue 250 completely through curved bone tunnel 280. Filament 270 can then be pushed downward, whether by a manual or automated mechanism internal or external to the device, in order to cause bight 275 to loosen and release the part of implant, suture, or tissue 250 that has been retrieved through curved bone tunnel 380.

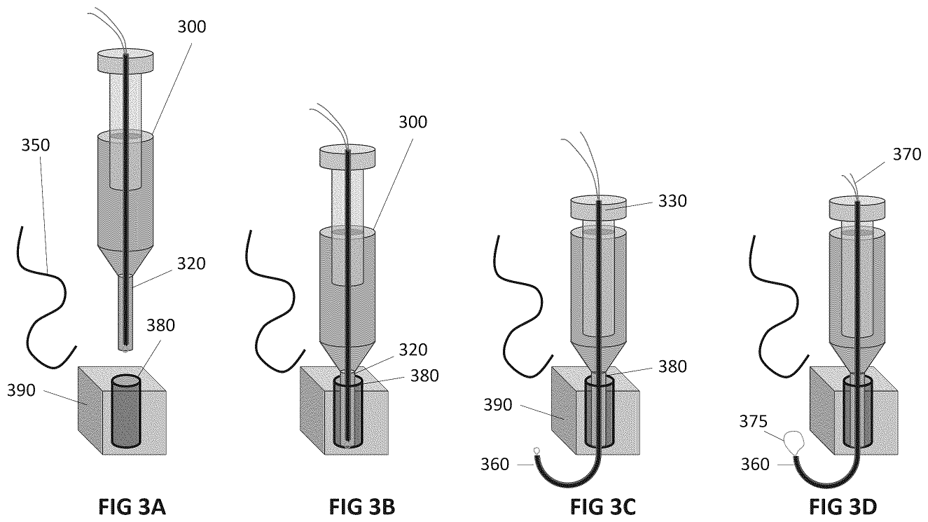

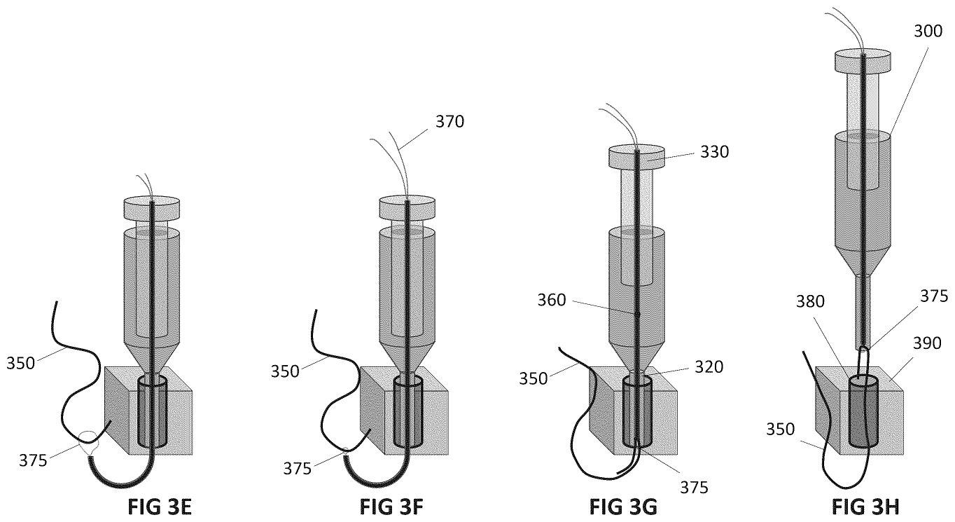

FIG. 3A-H depict the use of retrieval device 300 in another example application. Here, the retrieval device 300 is shown to retrieve one end of an implant, suture, or tissue 350 through an existing straight tunnel 380 within bone 390. FIG. 3A depicts the retrieval device 300, in the fully retracted configuration, outside of a region of bone 390 that possesses a straight bone tunnel 380. In FIG. 3B, the retrieval device 300, still in the fully retracted configuration, is positioned with the guide shaft 320 at least partially inserted through the straight bone tunnel 380. In FIG. 3C, downward force can be applied to plunger 330, whether by a manual or automated mechanism internal or external to the device, in order to cause tube 360, which can be comprised of superelastic material, to extend out from straight bone tunnel 380, underneath bone 390, and outward to a desired location. One of many reasons this approach may be advantageous is that the exit location of straight bone tunnel 380 can be inaccessible. In FIG. 3D, the two ends of filament 370 are pushed downward through tube 360, whether by a manual or automated mechanism internal or external to the device, in order to cause bight 375 to enlarge. In FIG. 3E, the implant, suture, or tissue 350 is manipulated by the user to cause one part of the implant, suture, or tissue to become pass through the opening formed by bight 375. In FIG. 3F, the two ends of filament 370 are pulled upward through tube 360, whether by a manual or automated mechanism internal or external to the device, in order to cause bight 375 to tighten around part of the implant, suture, or tissue 350 that was placed through the bight opening. In FIG. 3G, upward force is applied to plunger 330, whether by a manual or automated mechanism internal or external to the device, causing plunger 330 to slide upward and retracting tube 360 back into guide shaft 320, while bight 375 remains tightened around implant, suture, or tissue 350. In FIG. 3H, the retrieval device 300 is moved back out through straight bone tunnel 380, and away from bone 390, while bight 375 remains tightened around implant, suture, or tissue 350, in order to pull one end of implant, suture, or tissue 350 completely through straight bone tunnel 380. Filament 370 can then be pushed downward, whether by a manual or automated mechanism internal or external to the device, in order to cause bight 375 to loosen and release the part of implant, suture, or tissue 350 that has been retrieved through straight bone tunnel 380.

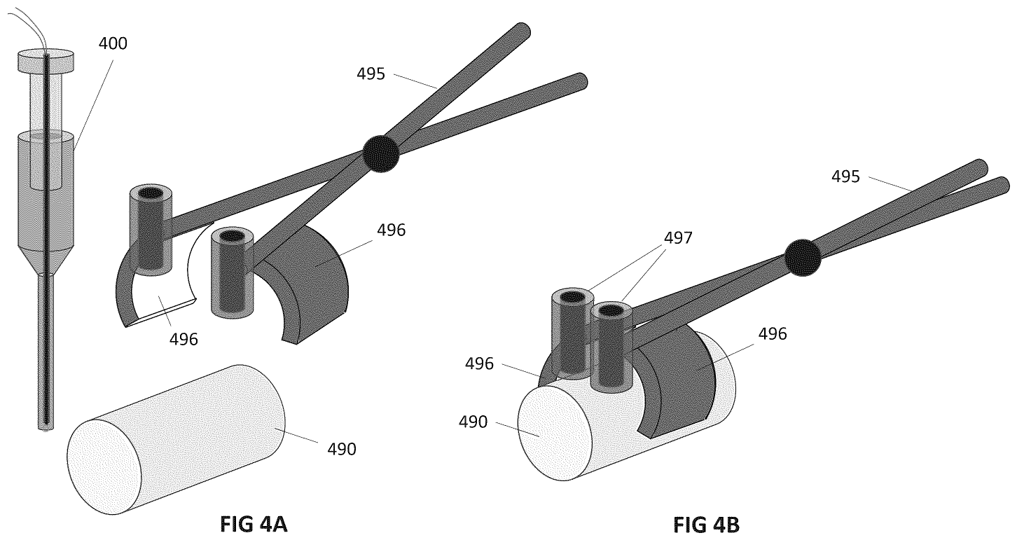

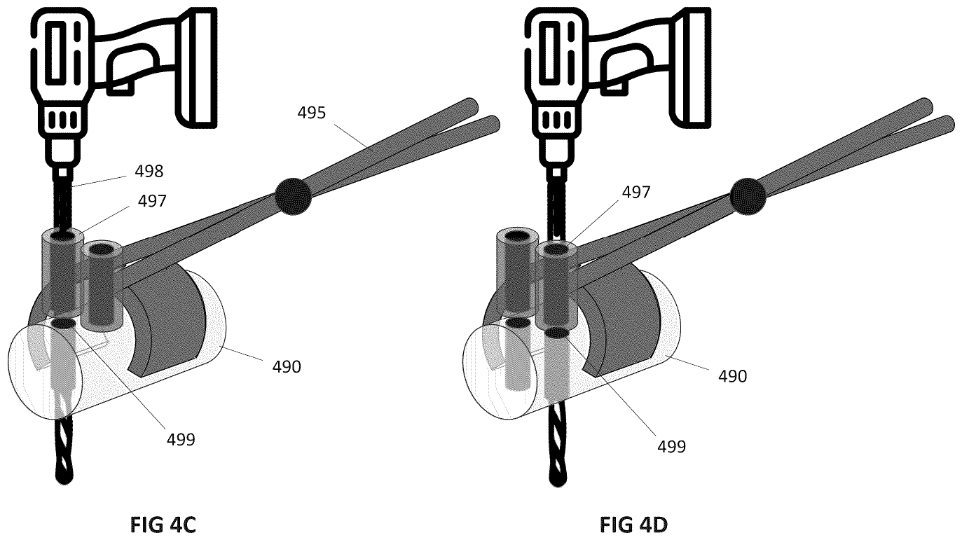

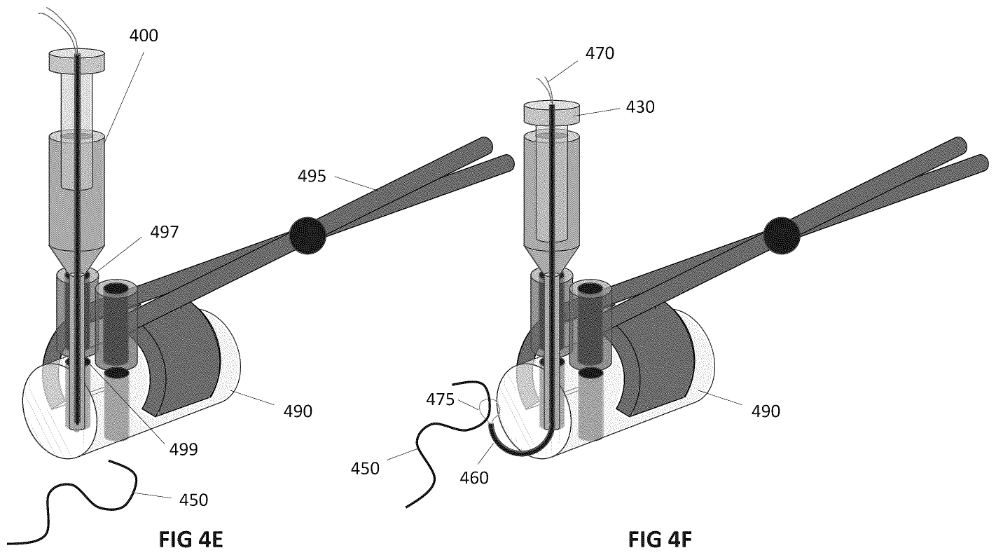

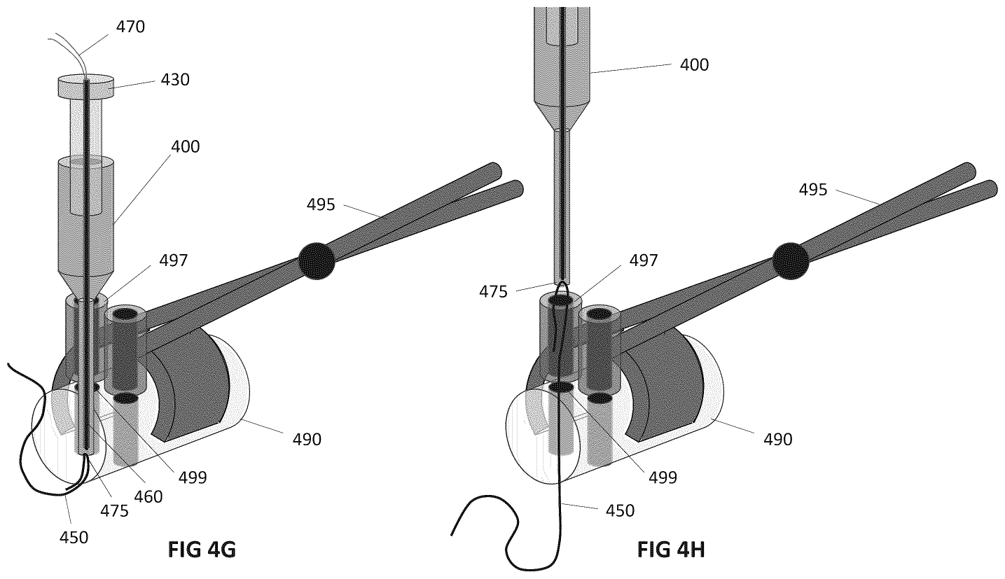

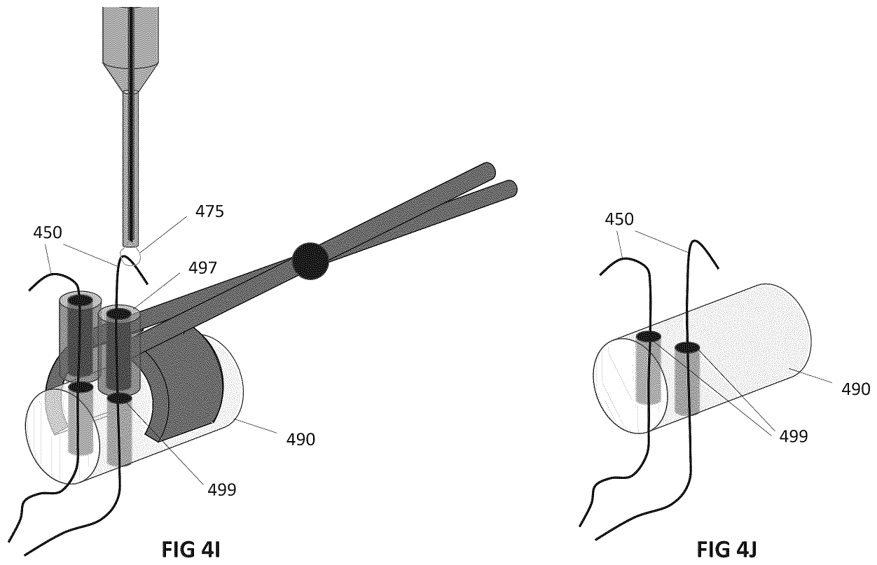

FIGS. 4A-J depict the use of retrieval device 400 in another example application. FIG. 4A shows the retrieval device 400, in the fully retracted configuration, a bone clamping device 495, and a bone 490 through which an implant, suture, or tissue will be passed. The bone clamping device 495 is depicted as being in an open position, where the arms 496 that interface with the bone are caused to spread apart. FIG. 4B depicts the bone clamping device 495 in the closed position, after capturing bone 490 between the arms 496 that interface with the bone, caused by scissoring action of the bone clamping device 495. The drill guide barrels 497, which possess cylindrical tunnels, are positioned above the sections of bone 490 where bone tunnels are desired to be created. In FIG. 4C, with bone clamping device 495 still securely holding bone 490, a drill with appropriately sized drill bit 498 is shown to pass through one of the drill guide barrels 497, in order to create one bone tunnel 499 at the desired location. FIG. 4D depicts repeating this step through the other drill guide barrel 497, in order to create a second bone tunnel 499 in bone 490. In FIG. 4E, with bone 490 still secure in clamping device 495, retrieval device 400 in fully retracted configuration is inserted through the first drill guide barrel 497 and through the first bone tunnel 499. Implant, suture, or tissue 450 is in a position that is ready for passing through bone 490. In FIG. 4F, with bone 490 still secure in clamping device 495, downward force can be applied to plunger 430, whether by a manual or automated mechanism internal or external to the device, in order to cause tube 460, which can be comprised of superelastic material, to extend outward to a desired location from underneath bone 490. FIG. 4F also shows the two ends of filament 470 having been pushed downward through tube 460, whether by a manual or automated mechanism internal or external to the device, in order to cause enlargement of bight 475, through which implant, suture, or tissue 450 can be manipulated to pass. In FIG. 4G, with bone 490 still secure in clamping device 495, filament 470 can be pulled upward through tube 460, whether by a manual or automated mechanism internal or external to the device, in order to cause bight 475 to tighten around part of the implant, suture, or tissue 450 that was placed through it. FIG. 4G also depicts plunger 430 having had an upward force applied to it, whether by a manual or automated mechanism internal or external to the device, causing plunger 430 to slide upward and retracting tube 460 back into retrieval device 400. In FIG. 4H, with bone 490 still secure in clamping device 495, retrieval device 400 is withdrawn from bone tunnel 499 and drill guide barrel 497, while implant, suture, or tissue 450 remains secure in bight 475. FIG. 4I depicts the steps shown in FIGS. 4E-H applied to the second drill guide barrel 497 and bone tunnel 499, resulting in one or more implants, sutures, or tissues 450 passing through bone 490. After the implant, suture, or tissue has been successfully passed through bone tunnel 499, bight 475 can be expanded to release it. FIG. 4J depicts bone 490 with clamping device 495 removed, and one or more implants, sutures, or tissues passing through bone tunnels 499. As those of skill in the art would appreciate, although FIGS. 4A-J depict an example wherein a retrieval device and bone clamping device were used in coordination to pass implants, sutures, or tissues through two bone tunnels, variations of these example embodiments can be used for just one or as many bone tunnels as a bone can accommodate.

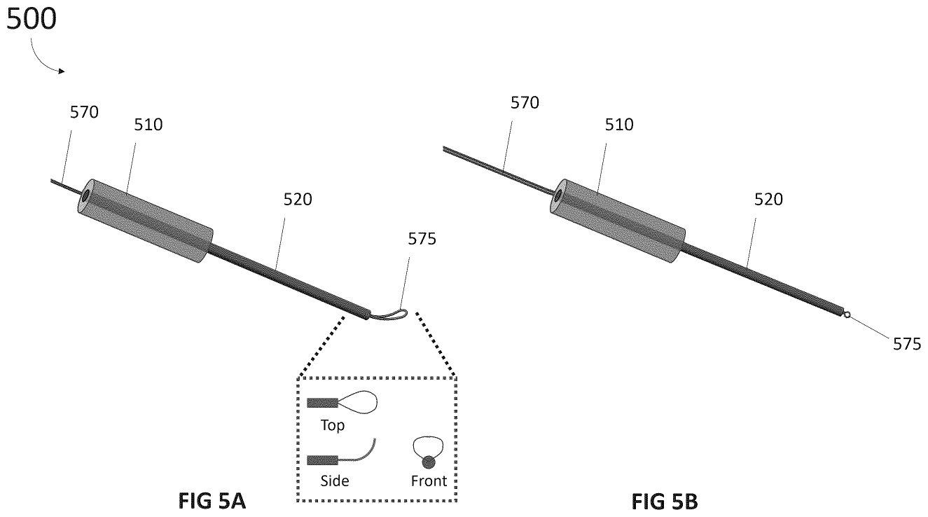

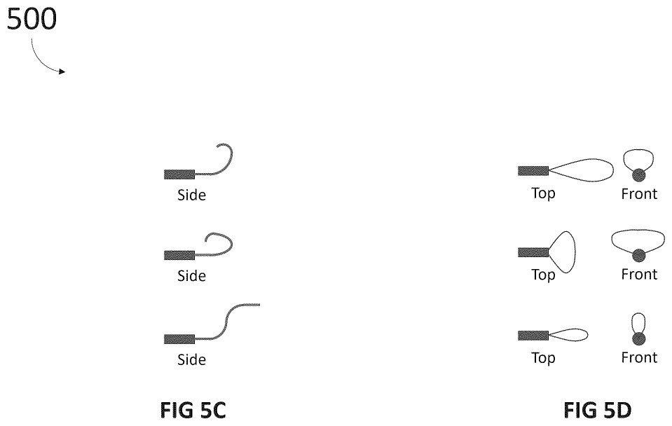

FIGS. 5A-B depict perspective views of another example embodiment of retrieval device 500 in extended and retracted configurations, respectively. According to one aspect of many embodiments, retrieval device 500 can comprise a housing 510, a guide shaft 520, and a filament 570. Housing 510 is rigidly attached to guide shaft 520. As depicted in FIG. 5A, within guide shaft 520 is a filament 570 whose two ends extend through and back out of housing 510 and guide shaft 520, thereby creating a bight 575 at the tip of guide shaft 520. In some embodiments, filament 570 can be comprised of a metal or polymer material that has been manufactured in such a way that bight 575 possesses a curved configuration, as detailed orthogonal views depict in the inset of FIG. 5A. As depicted in FIG. 5B, pulling forces applied to one or both ends of filament 570 causes it to slide within housing 510 and guide shaft 520, thereby retracting and shrinking the size of the opening formed by bight 575. Conversely, pushing forces applied to one or both ends of filament 570 causes it to slide within housing 510 and guide shaft 520, thereby extending and enlarging the size of the opening formed by bight 575 once again. Those of skill in the art will appreciate that filament 570 can comprise metal (e.g. nitinol), natural or synthetic material, organic or inorganic material, biodegradable or non-biodegradable polymer, or a combination thereof, in braided filament or monofilament configuration. Although FIG. 5A depicts bight 575 as having a single curve that subtends an angle of 90 degrees, those of skill in the art will also recognize that bight 575 can possess a curve comprising non-uniform curvatures, can be of any length, and can form an opening of any width. Example embodiments of various configurations of bight 575 having non-uniform curvatures are shown as side views in FIG. 5C. Example embodiments of various configurations of bight 575 being longer or forming openings of different widths are shown as top and front views in FIG. 5D.

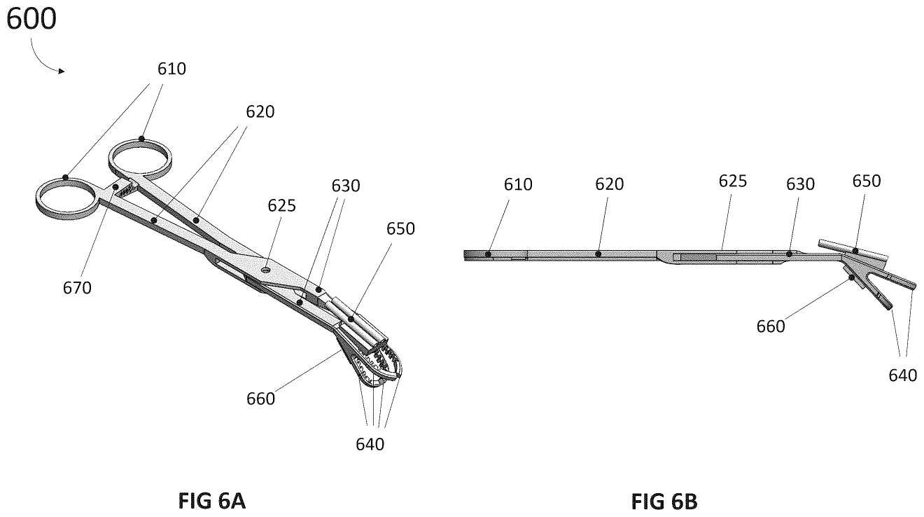

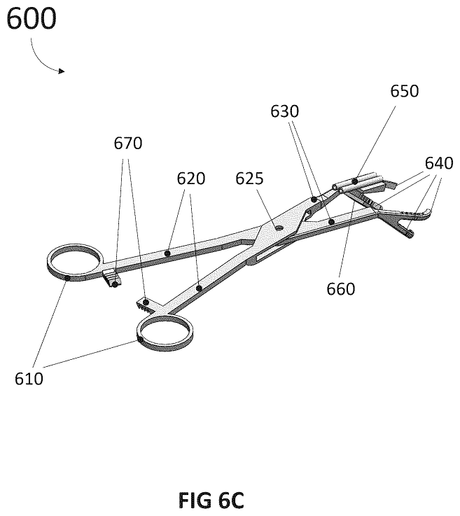

FIGS. 6A-C depict another example embodiment of bone clamping device 600 in various open and closed configurations. As depicted in FIG. 6A, bone clamping device 600 can be opened and closed by manipulation of two finger rings 610, each of which is connected to a shank 620. Ratcheting lock 670 enables bone clamping device 600 to remain securely closed without active force to tighten the clamp being applied to finger rings. The two shanks 620 cross at hinge 625, extending past hinge 625 to form the jaw elements 630. Moving the finger rings 610 closer together and farther apart cause the jaw elements 630 to open and close by way of a scissoring action. Serrated curved arms 640 are configured to interface with bone. As best seen in FIG. 6B, serrated curved arms 640 are oriented and curved in planes that lie at an angle relative to the plane of finger rings 610, shank 620, and jaw elements 630. Although bone clamping device 600 is shown to possess four serrated curved arms 640 such that each of the two pairs of arms are configured to interface directly opposite each other on either side of the bone, those of skill in the art will appreciate that there can be any number of arms oriented at any planar angle, with or without prescribed opposing alignment. As both FIGS. 6A and 6B show, a drill guide 650 comprising two cylindrical tunnels is attached near serrated curved arms 640. A bone support strut 660 is attached to the bottom side of bone clamping device 600, near serrated curved arms 640, and configured to interface with and stabilize the top of the bone when the bone is clamped between serrated curved arms 640. Although support strut 660 is depicted as a rectangular prism, those of skill in the art will appreciate that support strut 660 can be of any geometry and can possess curved edges and planes on any face. Those of skill in the art will also recognize that drill guide 650 can possess any number of cylindrical tunnels that can be mutually parallel or oblique, and can be oriented at any angle relative to serrated curved arms 640. As best seen in FIG. 6C, depicting the bone clamping device 600 in open configuration, although both drill guide 650 and bone support strut 660 are shown to be attached to the one of the jaw elements 630, those of skill in the art will appreciate that both drill guide 650 and bone support strut 660 can each be divided in two, whereby each half can be attached to one of the jaw elements 630.

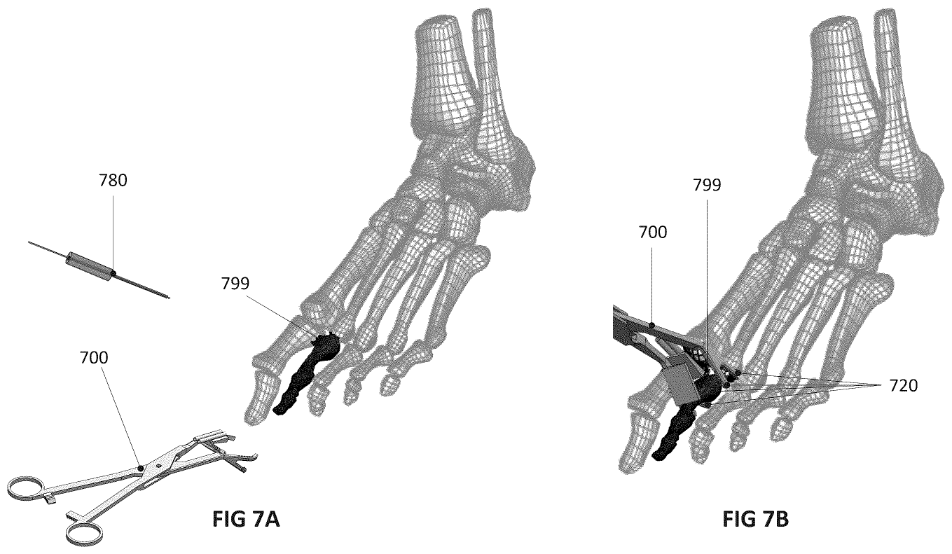

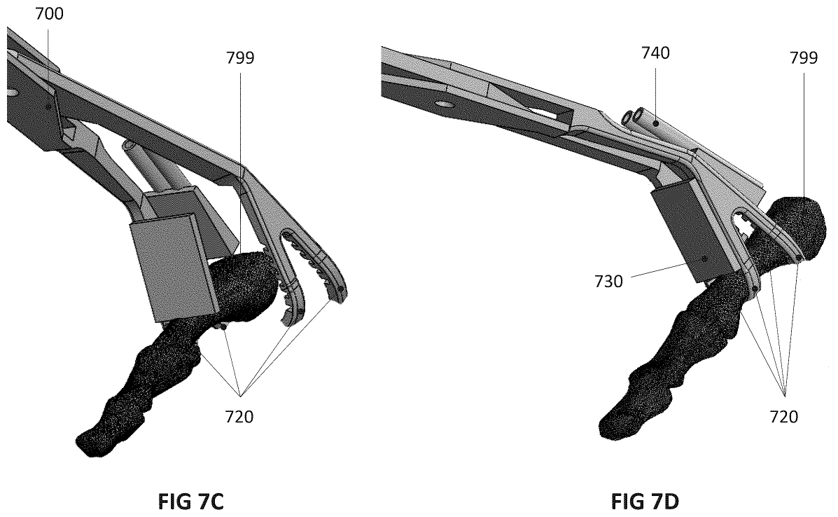

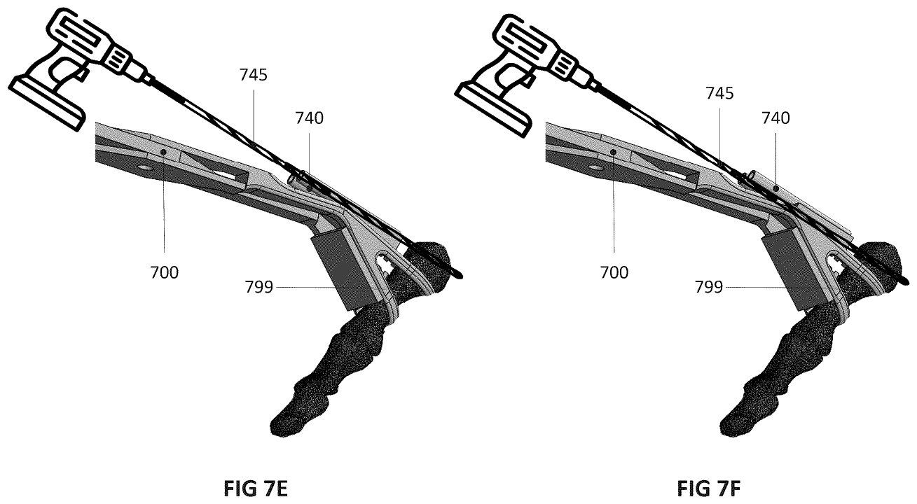

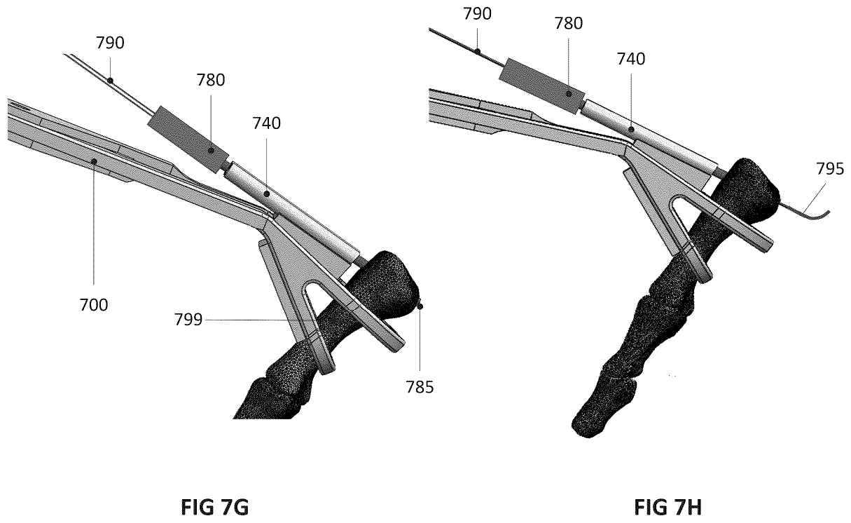

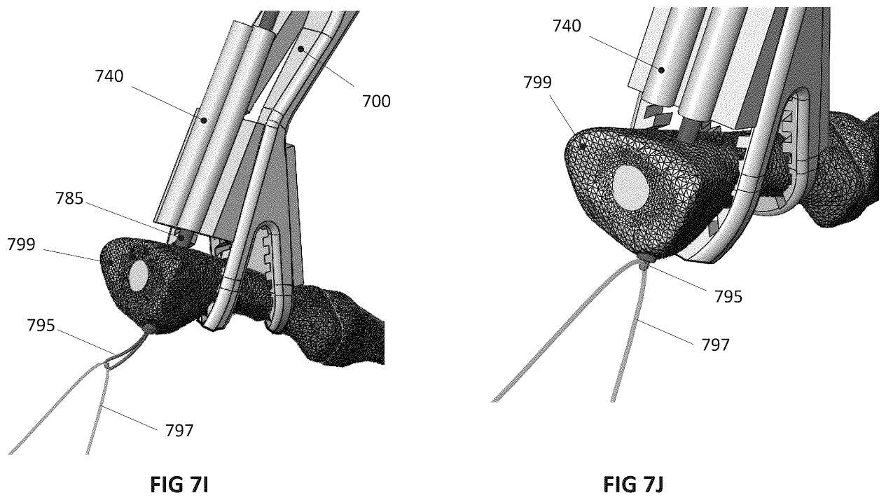

FIGS. 7A-J depict the method of using bone clamp 700 and retrieval device 780 in another example application. FIG. 7A shows a particular example in which bone clamp 700 and retrieval device 780 are to be used for passing an implant, suture, or tissue through one of the toe bones 799. As best seen in FIG. 7B, after preparation for the procedure, bone clamp 700 is manually caused to be in the open configuration and manually positioned to place serrated curved arms 720 around toe bones 799 by approaching from the top of the foot. FIGS. 7C-J depict toe bones 799 with other adjacent bones hidden, so as to improve clarity of illustration; it is to be understood that toe bones 799 remain attached to the rest of the body. FIG. 7C shows a close-up view of FIG. 7B, where serrated curved arms 720 of bone clamping device 700 are placed around toe bones 799, with other adjacent bones hidden. In FIG. 7D, bone clamping device 700 is manually caused to be in the closed configuration so that serrated curved arms 720 and bone support strut 730 are in contact with and securely tightened around the first bone of toe bones 799. The drill guide barrels 740, which possess cylindrical tunnels, are positioned above the sections of toe bones 799 where bone tunnels are desired to be created. As shown in FIG. 7E, with bone clamping device 700 still securely holding toe bones 799, a drill with appropriately sized drill bit 745 is shown to pass through one of the drill guide barrels 740, in order to create one bone tunnel at the desired location. The drill and drill bit 745 are removed from the first bone tunnel. FIG. 7F depicts repeating this step through the other drill guide barrel 740, in order to create a second bone tunnel. The drill and drill bit 745 are removed from the second bone tunnel. In FIG. 7G, while bone clamp 700 remains securely closed around toe bones 799, retrieval device 780 with filament 790 in the retracted configuration is inserted through one of the drill guide barrels 740, as well as its corresponding bone tunnel, such that the tip of the guide shaft 785 emerges from the bone tunnel exit. In FIG. 7H, a pushing force can be applied to filament 790 to extend and enlarge the size of bight 795, which has been manufactured in such a way that bight 795 possesses a curved configuration. As best seen in FIG. 7I from another perspective, while bone clamp 700 remains securely closed around toe bones 799 and guide shaft 785 remains in drill guide 740, a suture 797 can be inserted into bight 795. In FIG. 7J, a pulling force applied to filament 740 can cause bight 795 to tighten around and securely capture suture 797. While bone clamp 700 remains securely closed around toe bones 799, retrieval device 780 can be removed from toe bones 799 and drill guide 740, thereby pulling suture 797 through toe bones 799. Bone clamp 700 can then be caused to open in order to release toe bones 799, leaving only suture 797 passing through toe bones 799. Those of skill in the art will recognize that, although the method illustrated in FIGS. 7A-J depicts a specific example application of passing a suture through a toe bone using bone clamp 700 and retrieval device 780, they can also be used for passing implants, sutures, and tissues in different bones in other parts of the body.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1-11. (canceled)

12. An apparatus for clamping a bone material, the apparatus comprising:
a plurality of finger rings, each of which is coupled with a corresponding shank of a plurality of shanks, wherein the plurality of shanks is configured to transmit a manual force received from the plurality of finger rings;
a hinge coupled with the plurality of shanks, wherein the hinge comprises a fulcrum configured to create a scissoring action by the plurality of shanks;
a plurality of jaw elements disposed at a plurality of distal portions of the plurality of shanks, wherein the plurality of jaw elements is configured to transmit the manual force from the plurality of shanks to the bone material;
a plurality of serrated curved arms coupled with a plurality of distal portions of the plurality of jaw elements, the plurality of serrated curved arms configured to cradle and secure the bone material; and
a drill guide configured to control a position and a trajectory of a tunneling instrument for boring one or more tunnels through the bone material.

13. The apparatus of claim 12, wherein the device is configured to be opened and closed through a scissoring action actuated manually by the plurality of finger rings.

14. The apparatus of claim 12, wherein the drill guide comprises a plurality of cylindrical tunnels proximate to the plurality of serrated curved arms.

15. The apparatus of claim 14, wherein the cylindrical tunnels are mutually parallel.

16. The apparatus of claim 12, further comprising a ratchet lock configured to retain the apparatus in a closed position, while the apparatus is clamping the bone material, without requiring the manual force on the finger rings.

17. The apparatus of claim 12, wherein the plurality of serrated curved arms is disposed in a first plane at an angle relative to a second plane in which the plurality of finger rings, the plurality of shanks, and the plurality of jaw elements are disposed.

18. The apparatus of claim 12, wherein the plurality of finger rings comprises two finger rings, and wherein the plurality of shanks comprises two shanks.

19. The apparatus of claim 18, wherein the plurality of serrated curved arms comprises four serrated curved arms.

20. The apparatus of claim 12, further comprising a bone support strut configured to interface with and stabilize a top of the bone material when the bone material is clamped between the plurality of serrated curved arms.

21. A method for generating one or more bone tunnels configured for passage of one or more implants, sutures, or tissue therethrough, the method comprising:
closing a bone clamp around a bone material, wherein the bone clamp includes a drill guide configured to couple with a tunneling instrument;
creating, by the tunneling instrument, the one or more bone tunnels in the bone material, while the tunneling instrument is coupled with the drill guide of the bone clamp;
inserting at least a portion of a retrieval device through the drill guide of the bone clamp and the one or more bone tunnels, wherein the retrieval device includes a filament;
causing an opening of a bight, formed by the filament, to enlarge and shrink around the one or more implants, sutures, or tissue; and
retracting the retrieval device to cause the one or more implants, sutures, or tissue to pass into the one or more bone tunnels.

22. The method of claim 21, wherein the filament is configured to pass through the retrieval device.

23. The method of claim 21, further comprising disengaging the tunneling instrument from the drill guide of the bone clamp.

24. The method of claim 21, further comprising attaching the retrieval device to the drill guide of the bone clamp.

25. The method of claim 21, wherein the tunneling instrument comprises a drill bit.

26. The method of claim 21, wherein the tunneling instrument comprises a Kirschner wire.

27. The method of claim 21, wherein the filament comprises nitinol.

28. The method of claim 21, wherein the filament comprises nylon.

29. The method of claim 21, wherein the filament comprises polypropylene.
